(19) United States
(12) Patent Application Publication
(10) Pub. No.: US 2003/0114747 A1
(43) Pub. Date: Jun. 19, 2003

(54) Recanalization of occluded vessel using magnetic resonance guidance

(76) Inventor: Scott R. Smith, Chaska, MN (US)

Correspondence Address: Joseph R. Kelly, WESTMAN CHAMPLIN & KELLY, International Centre - Suite 1600, 900 South Second Avenue, Minneapolis, MN, 55402-3319, US

(21) Appl. No.: 10/017,213

(22) Filed: Dec. 14, 2001

(51) Int. Cl. A61B 5/05

(52) U.S. Cl. 600/420; 600/410; 600/423

(57) ABSTRACT

A method and apparatus for recanalizing a substantially totally occluded vessel in a subject. An image of the substantially totally occluded vessel is obtained using magnetic resonance. A recanalization device is guided using the obtained image. The occlusion is recanalized with the recanalization device. In one embodiment, a magnetic resonance signal is received with at least one external antenna located external to the body of the subject. A map image of the occluded vessel is generated using the signal received by the external antenna. A magnetic resonance signal is received with an internal antenna positioned within the body of the subject, proximate to the occluded vessel. The map image of the occluded vessel is locally enhanced using the signal received by the internal antenna.

### BACKGROUND OF THE INVENTION

The present invention relates generally to medical procedures in which a device is inserted into a body. More particularly, the present invention relates to medical procedures in which such a device is used while the body is in a magnetic resonance scanner.

The recanalization of a totally occluded artery is a difficult clinical challenge. Conventional X-ray angiography is unable to visualize the occluded portion of the artery. Furthermore, it is often difficult even to visualize the portion of the artery that is distal to the occlusion because of insufficient collateral flow. Previous attempts to design a device that automatically follows the occluded lumen have not been successful, primarily due to the complex, heterogeneous nature of the occlusive material and the lack of a distinct interface between the plaque and the vessel wall. Imaging solely from within the plaque using intravascular ultrasound (IVUS) or optical methods have failed because of poor penetration or a restricted field of view making the image difficult to interpret.

Tracking of catheters and other devices positioned within a body may be achieved by means of a magnetic resonance imaging system. Typically, such a magnetic resonance imaging system may be comprised of magnet means, pulsed magnetic field gradient generating means, a transmitter for electromagnetic waves in radio frequency (RF), a radio frequency receiver, a processor, and a controller. In a common implementation, an antenna is disposed either on the device to be tracked or on a guidewire or catheter used to assist in the delivery of the device to its destination. In one known implementation, the antenna comprises an electrically conductive coil that is coupled to a pair of elongated electrical conductors that are electrically insulated from each other. In one embodiment, the coil is arranged in a solenoid configuration. The patient is placed into the magnet means and the device is inserted into the patient. The magnetic resonance imaging system generates electromagnetic waves in radio frequency and magnetic field gradient pulses that are transmitted into the patient and that induce a resonant response signal from selected nuclear spins within the patient. This response signal induces current in the coil of electrically conductive wire attached to the device. The coil thus detects the nuclear spins in the vicinity of the coil. The radio frequency receiver receives this detected response signal and processes it and then stores it with the controller. This is repeated in three orthogonal directions. The gradients cause the frequency of the detected signal to be directly proportional to the position of the radio-frequency coil along each applied gradient.

The position of the radio frequency coil inside the patient may therefore be calculated by processing the data using Fourier transformations so that a positional picture of the coil is achieved. However, since the coil only reacts, the positional picture that is achieved is actually not a positional picture of the coil, but in fact a positional picture of the position of the response signals inside the patient. Since this positional picture contains no information on the region beyond the immediate vicinity of the coil, in one implementation this positional picture is superposed with a magnetic resonance image of the region of interest. This picture of the region may be taken and stored at the same occasion as the positional picture or at any earlier occasion.

Other types of antennas for intravascular devices are known, in addition to the above-mentioned coil-type antenna. One such antenna simply consists of a loop of electrically conductive material coupled to the ends of two elongated electrical conductors that are electrically insulated from each other. Radio frequency antennas in the form of a coil couple inductively to the electromagnetic field and they allow obtaining a substantially spatially uniform magnetic field which results in a relatively uniform image intensity over a wide region. However, coil configurations are bulky (the received signal is determined by the loop diameter) and cannot be implemented for use in narrow vessels, whereby their use for the placement of medical appliances such as catheters may be critical. Furthermore, the spot image which is provided for by the coil antenna does not allow knowing or even evaluating the orientation of the device. As a result, the magnetic resonance imaging system cannot be used for steering the device into tortuous areas such as blood vessels.

Another antenna configuration, sometimes referred to as an open wire length antenna, comprises first and second elongated electrical conductors that are electrically insulated from each other and have spaced-apart distal ends. As opposed to the coil configuration, the open wire length antenna couples capacitively to the electromagnetic field and as the received signal originates from the immediate neighborhood of the open wire length, it becomes possible to obtain an image of the antenna, of its position, as well as of its orientation. This makes steering of the appliance possible. The open wire length antenna may be extremely thin and it may also have a high flexibility, allowing safe driving and passage through vascular configurations, even in tortuous and restricted areas thereof. This facilitates the use of magnetic resonance imaging procedures in interventional conditions where time and precision are of the essence. By repeatedly measuring, reconstructing, and displaying the image with a very short image repetition time, a magnetic resonance imaging fluoroscopy system can be created. The open wire length antenna can also be used to make a high resolution image of a vessel wall.

In one embodiment, the open wire length antenna may be formed by a coaxial cable having first and second conductors arranged in a coaxial configuration. In another embodiment, the open wire length antenna may be made of a coaxial cable in which the shield and insulators are respectively made of a conductor coating and insulating coatings. The open wire length antenna may also be made of two conducting strands insulated from one another, twisted or parallel to one another. The open wire length antenna may be included in a catheter and the like. As opposed to coil antennas for which the received signal depends on the loop diameter, the diameter of the open wire length antenna is of secondary relevance and, therefore, the open wire length antenna may be devised to form the whole or part of a guidewire as used in vascular procedures for the positioning of catheters and the like.

The above-described methods and apparatus have not previously been employed to recanalize a totally occluded vessel.

The present invention provides a solution to this and other problems and offers other advantages over the prior art.

### SUMMARY OF THE INVENTION

One embodiment of the present invention is directed to a method of recanalizing a substantially totally occluded vessel in a subject. Pursuant to the method, an image of the substantially totally occluded vessel is obtained using magnetic resonance. A recanalization device is guided through the vessel using the obtained image. The occlusion is recanalized with the recanalization device.

In one embodiment of the above method, a magnetic resonance signal is received with at least one external antenna located external to the body of the subject. A map image of the occluded vessel is generated using the signal received by the external antenna. A magnetic resonance signal is received with an internal antenna positioned within the body of the subject, proximate to the occluded vessel. The map image of the occluded vessel is locally enhanced using the signal received by the internal antenna.

In another embodiment of the above method, a magnetic resonance signal is received with at least one external antenna located external to the body of the subject. A map image of the occluded vessel is generated using the signal received by the external antenna. A magnetic resonance signal is received with an internal antenna positioned within the body of the subject, proximate to the occluded vessel. A local image of the occluded vessel is generated using the signal received by the internal antenna.

Another embodiment of the present invention is directed to an apparatus for imaging an occluded vessel in a subject. The apparatus includes a magnetic field generator, a magnetic field gradient generator, a radio frequency (RF) signal generator, an external RF receiver, an internal RF antenna, a controller and a visual display. The magnetic field generator establishes a magnetic field on the subject. The magnetic field gradient generator establishes gradients in the magnetic field. The RF signal generator emits pulsed RF signals to at least the occluded vessel of the subject. The external RF receiver is positioned external to the body of the subject. The external RF receiver receives signals emitted from the subject in response to the RF pulses and provides an output signal in response to the received signals. The internal RF antenna is adapted to be positioned in the occluded vessel proximate the occlusion, to receive RF signals emitted from the subject in response to the RF pulses and to provide an output signal in response to the received signals. The controller receives and processes the output signals from the external RF receiver and internal RF antenna and produces magnetic resonance (MR) information related thereto. The visual display receives the MR information produced by the controller and displays the MR information as an image of the occluded vessel.

In one embodiment of the above-described apparatus, an open wire length antenna is employed as the internal antenna. The open wire length antenna includes first and second elongated electrical conductors that are electrically insulated from each other and have spaced-apart distal ends that can be positioned proximate the occlusion. In an illustrative embodiment, the first conductor of the internal antenna is adapted to function as an ablation wire in addition to its role in receiving the magnetic resonance signal. In this embodiment, the first conductor has an uninsulated distal tip that can be positioned proximate the occlusion. The first conductor receives and conducts an electrical ablation current such that the distal tip of the conductor vaporizes the substance forming the occlusion. In one embodiment, the first conductor is couplable to an ablation power supply that applies the electrical ablation current to the first conductor. An illustrative embodiment further includes a switch adapted to selectably switch the first conductor between the processor and the ablation power supply. In one embodiment, the open wire length antenna comprises a coaxial cable with the first conductor being the center conductor of the coaxial cable.

These and various other features as well as advantages which characterize the present invention will be apparent upon reading of the following detailed description and review of the associated drawings.

### BRIEF DESCRIPTION OF THE DRAWINGS

FIG. 1 is a partial block diagram of a magnetic resonance imaging and intravascular guidance system according to an illustrative embodiment of the present invention.

FIG. 2 is a flow chart representing a method of recanalizing a substantially totally occluded vessel in a subject according to an illustrative embodiment of the present invention.

FIG. 3 is a schematic diagram of a recanalization device according to an illustrative embodiment of the present invention.

FIG. 4 is a flow chart representing a method of obtaining an image of a substantially totally occluded vessel in a subject according to an illustrative embodiment of the present invention.

FIG. 5a is a cross-sectional view of an intravascular device according to an illustrative embodiment of the present invention.

FIG. 5b is a side view of an intravascular device according to an illustrative embodiment of the present invention.

FIG. 6 is a cross-sectional side view of an intravascular coil device according to an illustrative embodiment of the present invention.

FIG. 7 is a flow chart representing a method of obtaining an image of a substantially totally occluded vessel in a subject according to an illustrative embodiment of the present invention.

### DETAILED DESCRIPTION OF ILLUSTRATIVE EMBODIMENTS

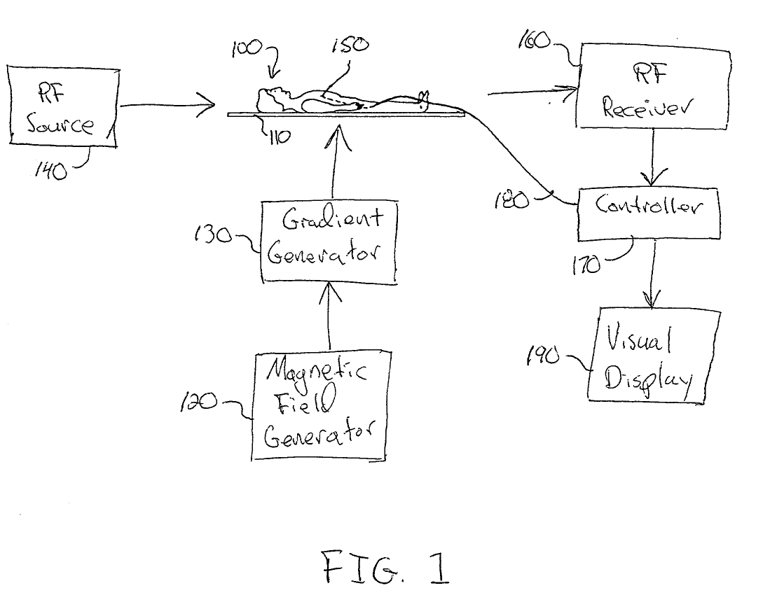

FIG. 1 is a partial block diagram of a magnetic resonance imaging and intravascular guidance system according to an illustrative embodiment of the present invention. In FIG. 1, subject 100 on support table 110 is placed in a homogeneous magnetic field generated by magnetic field generator 120. Magnetic field generator 120 typically comprises a cylindrical magnet adapted to receive subject 100. Magnetic field gradient generator 130 creates magnetic field gradients of predetermined strength in three mutually orthogonal directions at predetermined times. Magnetic field gradient generator 130 is illustratively comprised of a set of cylindrical coils concentrically positioned within magnetic field generator 120. A region of subject 100 into which a device 150, shown as a catheter, is inserted, is located in the approximate center of the bore of magnet 120.

RF source 140 radiates pulsed radio frequency energy into subject 100 and the MR active sample within device 150 at predetermined times and with sufficient power at a predetermined frequency to nutate nuclear magnetic spins in a fashion well known to those skilled in the art. The nutation of the spins causes them to resonate at the Larmor frequency. The Larmor frequency for each spin is directly proportional to the strength of the magnetic field experienced by the spin. This field strength is the sum of the static magnetic field generated by magnetic field generator 120 and the local field generated by magnetic field gradient generator 130. In an illustrative embodiment, RF source 140 is a cylindrical external coil that surrounds the region of interest of subject 100. Such an external coil can have a diameter sufficient to encompass the entire subject 100. Other geometries, such as smaller cylinders specifically designed for imaging the head or an extremity can be used instead. Non-cylindrical external coils such as surface coils may alternatively be used.

Device 150 is inserted into subject 100 by an operator. Device 150 may be a guide wire, a catheter, an ablation device or a similar recanalization device. Device 150 includes an RF antenna which detects MR signals generated in both the subject and the device 150 itself in response to the radio frequency field created by RF source 140. Since the internal device antenna is small, the region of sensitivity is also small. Consequently, the detected signals have Larmor frequencies which arise only from the strength of the magnetic field in the proximate vicinity of the antenna. The signals detected by the device antenna are sent to imaging and tracking controller unit 170 via conductor 180.

External RF receiver 160 also detects RF signals emitted by the subject in response to the radio frequency field created by RF source 140. In an illustrative embodiment, external RF receiver 160 is a cylindrical external coil that surrounds the region of interest of subject 100. Such an external coil can have a diameter sufficient to encompass the entire subject 100. Other geometries, such as smaller cylinders specifically designed for imaging the head or an extremity can be used instead. Non-cylindrical external coils, such as surface coils, may alternatively be used. External RF receiver 160 can share some or all of its structure with RF source 140 or can have a structure entirely independent of RF source 140. The region of sensitivity of RF receiver 160 is larger than that of the device antenna and can encompass the entire subject 100 or a specific region of subject 100. However, the resolution which can be obtained from external RF receiver 160 is less than that which can be achieved with the device antenna. The RF signals detected by external RF receiver 160 are sent to imaging and tracking controller unit 170 where they are analyzed together with the RF signals detected by the device antenna.

The position of device 150 is determined in imaging and tracking controller unit 170 and is displayed on display means 180. In an illustrative embodiment of the invention, the position of device 150 is displayed on display means 180 by superposition of a graphic symbol on a conventional MR image obtained by external RF receiver 160. Alternatively, images may be acquired with external RF receiver 160 prior to initiating tracking and a symbol representing the location of the tracked device be superimposed on the previously acquired image. Alternative embodiments of the invention display the position of the device numerically or as a graphic symbol without reference to a diagnostic image.

In an illustrative embodiment of the present invention, device 150 is a recanalization device adapted to recanalize an occluded vessel. One embodiment of the present invention is directed toward a method of recanalizing a totally occluded vessel. Recanalization of a totally occluded vessel is a difficult task because visualization of the totally occluded vessel is very difficult. Without proper visualization of the occluded vessel, guidance of the recanalization device is extremely difficult. Conventional X-ray angiography is unable to visualize the occluded portion of the artery. Also, it is difficult to visualize the vessel distal to the occlusion because of insufficient collateral flow. Previous attempts to design a device that automatically follows the occluded lumen have not been successful, primarily due to the complex, heterogeneous nature of the occlusive material and the lack of a distinct interface between the plaque and the vessel wall. Imaging solely from within the plaque using intravascular ultrasound (IVUS) or optical methods have failed because of poor penetration or a restricted field of view making the image difficult to interpret.

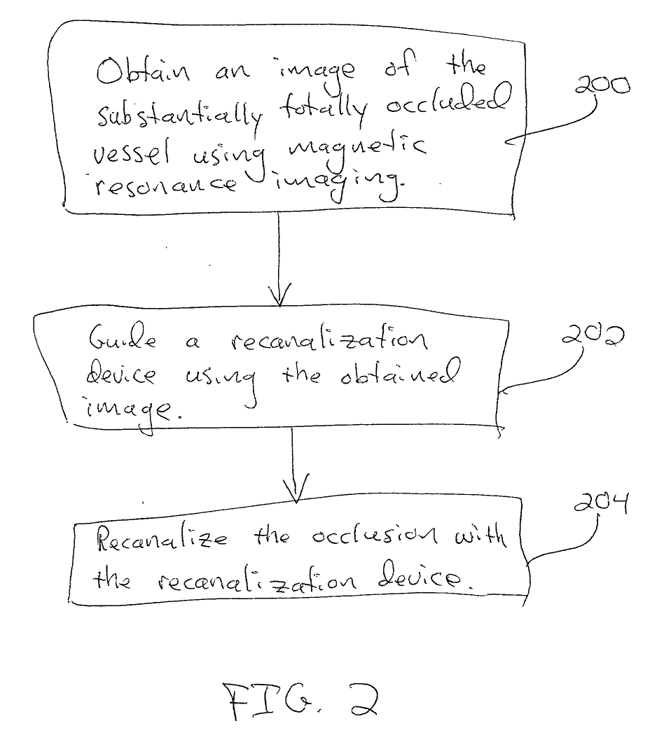

FIG. 2 is a flow chart representing a method of recanalizing a substantially totally occluded vessel in a subject according to an illustrative embodiment of the present invention. At step 200, an image of the substantially totally occluded vessel is obtained using magnetic resonance. At step 202, a recanalization device is guided through the vessel using the obtained image. At step 204, the occlusion is recanalized with the recanalization device. In an illustrative embodiment of the method represented in FIG. 2, step 200 comprises obtaining an image of an occluded portion of the vessel using magnetic resonance.

In one embodiment of the method of FIG. 2, the image includes an indication of a position of the recanalization device with respect to the occluded vessel. In a further embodiment, the image further includes an indication of a spatial orientation of the recanalization device with respect to the occluded vessel. In another embodiment, the image includes an image of the recanalization device.

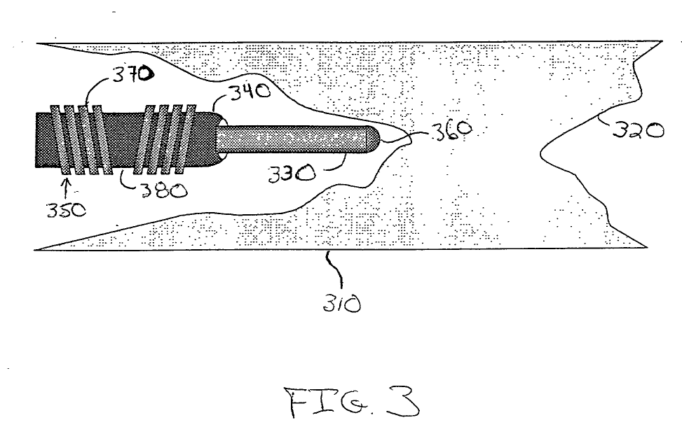

In an illustrative embodiment of the method of FIG. 2, the occluded vessel is recanalized using an ablation device. FIG. 3 is a schematic diagram of a recanalization device 330 according to an illustrative embodiment of the present invention. FIG. 3 shows a vessel 310 totally occluded by occlusion 320. Recanalization device 330 is an ablation device. The distal tip 360 of a core wire in ablation device 330 is exposed. In operation of the illustrative embodiment shown in FIG. 3, ablation device 330 is advanced through catheter 340 deployed in the occluded vessel 310 until the distal tip 360 of the core wire of ablation device 330 is disposed proximate the occlusion 320. An electrical current is applied to the core wire of ablation device 330 such that the distal tip 360 of the conductor heats and possibly vaporizes the substance forming the occlusion 320. In an illustrative embodiment, the electrical current applied to the ablation device 330 is a radio frequency current.

While this discussion has proceeded with respect to an ablation device and using electrical current, other devices could be used as well. Such devices may use other energy sources such as ultrasound, laser energy, or they may simply involve the use of a stiff pointer wire.

Antenna 350 receives magnetic resonance signals generated in the subject in response to the radio frequency field generated by RF source 140. Antenna 350 shown in FIG. 3 is an opposed solenoid coil antenna. However, any other intravascular antenna configuration may also be employed in accordance with the present invention.

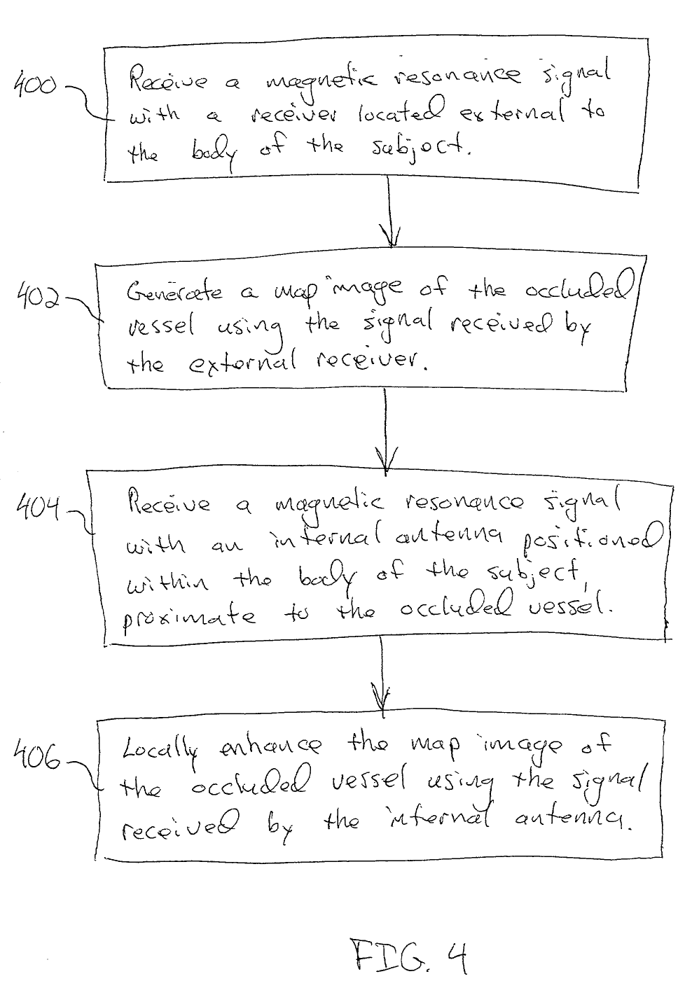

In one embodiment of the method represented in FIG. 2, obtaining the image (step 200) is achieved by performing the steps set out in the flow chart of FIG. 4. At step 400 of FIG. 4, a magnetic resonance signal is received with an external receiver 140 located external to the body of the subject. At step 402, a map image of the occluded vessel is generated using the signal received by the external receiver 140. At step 404 a magnetic resonance signal is received with an internal antenna, such as antenna 350, positioned within the body of the subject, proximate to the occluded vessel. In an illustrative embodiment, the magnetic resonance signals received by external receiver 140 and internal antenna 350 comprise radio frequency signals that are representative of the magnetic resonance of atomic particles in a vicinity proximate to the corresponding antenna. At step 406, the map image of the occluded vessel is locally enhanced using the signal received by internal antenna 350.

In an illustrative embodiment of the present invention, the map image of the occluded vessel is generated prior to guiding step 202 and recanalizing step 204 of FIG. 2. The map image is then locally enhanced in real time using the signal received by internal antenna 350. Recanalization device 330 is then guided using the locally enhanced map image. In a further illustrative embodiment, locally enhancing step 406 is achieved by generating a local image of the occluded vessel using the signal received by internal antenna 350 and then superimposing the local image on the map image generated using the signal received by external receiver 140.

In an illustrative embodiment of the present invention, the internal antenna is integral with equipment deployed in the vessel to assist in the delivery of the recanalization device 300 to the occlusion, such as a catheter 340, as shown in FIG. 3, or a guidewire. However, in an alternative embodiment, the internal antenna is integral with the recanalization device 330.

In an illustrative embodiment of the present invention a position of the recanalization device 330 is calculated based upon the magnetic resonance signal received by the internal antenna. In a further illustrative embodiment, an integrated image of the occluded vessel is generated based upon the map image, the locally enhanced image, and the calculated position of the recanalization device 330. The integrated image is displayed on visual display 190. This integrated image illustratively comprises a three-dimensional rendering showing the recanalization device 330 and the occluded vessel 310. In an illustrative embodiment, an integrated image of the occluded vessel 310 is generated based upon the map image and the locally enhanced image. A symbol is then superimposed on the integrated image at a position representing the calculated position of the recanalization device 330.

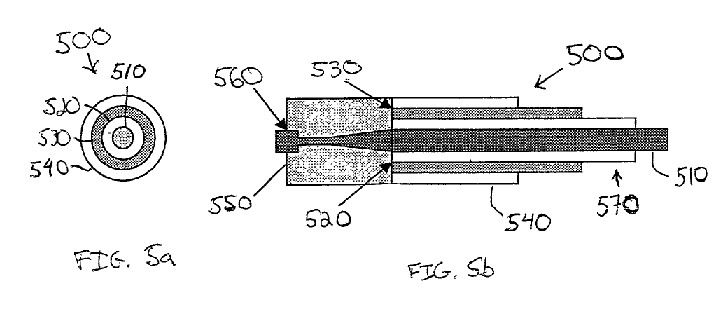

FIGS. 5a and 5b show an intravascular device 500 according to an illustrative embodiment of the present invention. FIG. 5a is a cross-sectional view of intravascular device 500. FIG. 5b is a side view of intravascular device 500. Intravascular device 500 includes center wire 510, inner insulating layer 520, shield 530, outer insulating layer 540, and distal insulator 550. Center wire 510 is comprised of an electrically conductive material. In an illustrative embodiment, the conductive and insulating layers 510, 520, 530 and 540 are staggered at the proximal end 570 to permit coupling of device 500 to a connector cable 180 attached to an antenna input to imaging controller 170. In an illustrative embodiment, center wire 510 is comprised of nitinol. In a further illustrative embodiment, center wire 510 is gold-plated to increase its conductivity. Inner insulating layer 520 is illustratively comprised of polyamide, polytetraflouroethylene (PTFE, or teflon) or parylene. Shield 530 is comprised of an electrically conductive material, illustratively gold. Outer insulating layer 540 is comprised of PTFE in an illustrative embodiment. Distal insulator 550 is comprised of a soft insulating material, illustratively silicone or polyurethane.

Intravascular device 500 functions as an open wire length antenna. An open wire length antenna includes an open-ended or undelimited piece of wire, as opposed to a closed wire length such as a piece of wire with a coil configuration at the end. As opposed to a coil configuration, the open wire length antenna couples capacitively to the electromagnetic field. The antenna receives signals that originate from the immediate neighborhood of the open wire length. Using this signal obtained by intravascular device 500, imaging controller 170 generates an image of the antenna, its position and its orientation. In an illustrative embodiment of the present invention, the signal received by intravascular device 500 is also used by imaging controller 170 to generate an image of the tissue surrounding the device 500, including an image of the vessel and the occlusion. These images can be used to assist the operator in steering device 500 during a recanalization procedure. In contrast to coil antennas for which the received signal depends on the loop diameter, the diameter of the open wire length antenna is of secondary relevance. The open wire length configuration of device 500 allows it to be extremely thin and to have a high flexibility, allowing safe driving and passage through vascular configurations, even in tortuous and restricted areas thereof. This opens the way to using magnetic resonance imaging procedures in interventional conditions where time and precision are of the essence. In an illustrative embodiment, an image is generated and displayed with a very short image repetition time.

In an illustrative embodiment of the present invention, intravascular device 500 functions as an ablation device in addition to its role as an antenna. In such an embodiment, the distal end 560 of center wire 510 is exposed, as shown in FIG. 5b. In operation, the center conductor 510 is positioned in the occluded vessel 300 with the distal tip 560 proximate the occlusion 320. An electrical ablation current is applied to center conductor 510 such that the distal tip 560 of center conductor 510 heats up and vaporizes the substance forming the occlusion 320. In an illustrative embodiment, the electrical ablating energy is delivered on demand by switching center conductor 510 between imaging controller 170 and an ablation power supply. Such a switch is illustratively located somewhere along the length of connector cable 180.

In an alternative embodiment of the present invention, intravascular device 500 is a guidewire adapted to assist in the delivery of a recanalization device to the occlusion site 320.

In FIG. 5, an open wire length antenna is implemented using a coaxial cable configuration. In an illustrative embodiment of the present invention, the open wire length antenna may be made of a coaxial cable in which the shield and insulators are respectively made of a conductor coating and insulating coatings. Other open wire length configurations may also be employed in accordance with the present invention. In one embodiment, the open wire length antenna is made of two conducting strands insulated from one another, twisted or parallel to one another.

In an illustrative embodiment of the present invention, an intravascular device that functions as an open wire length antenna, such as intravascular device 500 in FIG. 5, is used in conjunction with a second internal antenna in order to collect more information about the surroundings of the intravascular device, thereby facilitating a clearer and more detailed view of the vessel, the occlusion and the device. For example, in one embodiment, intravascular device 500 is used in conjunction with a catheter 340 that includes an antenna 350 as shown in FIG. 3. Antenna 350 of FIG. 3 has a solenoid configuration. Solenoid antenna 350 includes a transmission line that is comprised of two elongated electrical conductors that are electrically insulated from each other. These electrical conductors are integral with catheter 340 and are not shown in FIG. 3. The distal ends of the conductors are coupled to each other via a coil 370 comprised of a helically wound electrical conductor as shown in FIG. 3. In operation, the coil 370 is positioned proximate the occlusion to receive magnetic resonance signals given off by the surrounding tissue. The particular solenoid antenna shown in FIG. 3 is an opposed solenoid configuration. Illustratively, in such a configuration, one conductor of the transmission line couples to a first end of opposed solenoid coil 370. Opposed solenoid coil 370 is wound around catheter 340 in one direction. A gap 380 is formed and the opposed solenoid coil 370 is wound in the opposite direction around catheter 340. A conductor (not shown) coupled to the second end of opposed solenoid coil 370 couples to the second conductor of the transmission line. In an illustrative embodiment, opposed solenoid coil 370 is wound approximately 10-12 turns around catheter 340 in each direction.

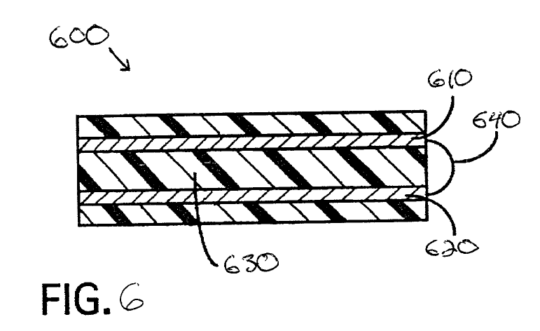

FIG. 6 is a cross-sectional side view of an intravascular coil device 600 according to another embodiment of the present invention. Intravascular coil device 600 has a pair of electrodes 610, 620 which, in the form shown, are generally parallel and spaced from each other. Coil device 600 has a dielectric material 630 which serves to reduce dielectric losses of the coil device 600. Ends of conductors 610, 620 are electrically connected by wire 640. In operation, connecting wire 640 is positioned in the occluded vessel 310 proximate occlusion 320 to receive magnetic resonance signals emitted by the surrounding tissue, including the vessel wall 310 and occlusion 320.

In an illustrative embodiment of the present invention, intravascular coil device 600 is implemented as a guidewire used to assist in the delivery of recanalization device 330 to the site of occlusion 320. In an alternative embodiment, coil device 600 is an independent device used in conjunction with recanalization device 330 for imaging the recanalization device 330 and the surrounding tissue and/or positioning recanalization device 330. In one implementation of this embodiment, recanalization device 330 and coil device 600 are both advanced through a catheter 340 and positioned proximate occlusion 320. In another alternative embodiment, a coil device similar to coil device 600 is implemented as a catheter such as catheter 340 in FIG. 3. In one implementation of this embodiment, the conductors 610 and 620 are conductive layers separated by an insulating layer and the distal ends of the conductive layers are electrically coupled in a manner similar to the device shown in FIG. 6. In another implementation wherein a coil device is implemented as a catheter, the conductors 610 and 620 are wire conductors embedded in the catheter wall and separated by an insulating material, the distal ends of the wire conductors being electrically coupled in a manner similar to the device shown in FIG. 6. In another alternative embodiment of the present invention, coil device 600 is integral with, or integrally connectable to, recanalization device 330.

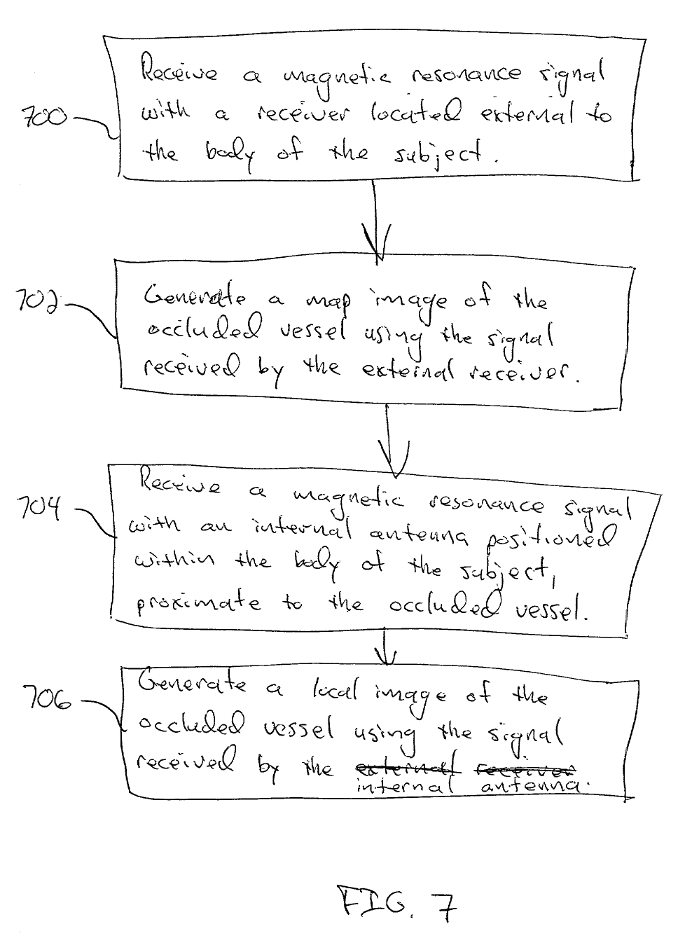

In one embodiment of the method represented in FIG. 2, obtaining the image (step 200) is achieved by performing the steps set out in the flow chart of FIG. 7. At step 700 of FIG. 7, a magnetic resonance signal is received with an external receiver 140 located external to the body of the subject. At step 702, a map image of the occluded vessel is generated using the signal received by the external receiver 140. At step 704 a magnetic resonance signal is received with an internal antenna, such as antenna 350, positioned within the body of the subject, proximate to the occluded vessel. In an illustrative embodiment, the magnetic resonance signals received by external receiver 140 and internal antenna 350 comprise radio frequency signals that are representative of the magnetic resonance of atomic particles in a vicinity proximate to the corresponding antenna. At step 406, a local image of the occluded vessel is generated using the signal received by internal antenna 350.

In an illustrative embodiment of the present invention, the map image generated in step 702 is combined with the local image generated in step 706 to generate an integrated image of the occluded vessel. In one embodiment, a position of the recanalization device 330 is calculated based on the magnetic resonance signal received by internal antenna 350. The calculated position is then used together with the map image and the local image to generate the integrated image of the occluded vessel. In one implementation, the integrated image is generated by superimposing the local image on the map image.

In an illustrative embodiment of the present invention, the map image of the occluded vessel is generated prior to guiding step 202 and recanalizing step 204 of FIG. 2. The local image is then generated in real time using the signal received by the internal antenna 350. Recanalization device 330 is then guided in real time using the local image.

It should also be noted that internal antenna can be disposed on a wire over which, or a sleeve through which, recanalization device 330 travels. The sleeve can serve shielding purposes (such as operate as a primary or secondary shield) for the device as well.

It is to be understood that even though numerous characteristics and advantages of various embodiments of the present invention have been set forth in the foregoing description, together with details of the structure and function of various embodiments of the invention, this disclosure is illustrative only, and changes may be made in details, especially in matters of structure and arrangement of parts within the principles of the present invention to the full extent indicated by the broad general meaning of the terms in which the appended claims are expressed. For example, the recanalization of a totally occluded vessel using MR guidance according to the present invention may be employed with MR systems that employ transmission signals having frequencies other than radio frequency. Other modifications can also be made.

What is claimed is:

1. A method of recanalizing a substantially totally occluded vessel in a subject, comprising steps of:
(a) obtaining an image of the substantially totally occluded vessel using magnetic resonance;
(b) guiding a recanalization device using the obtained image; and
(c) recanalizing the occlusion with the recanalization device.

2. The method of claim 1 wherein obtaining step (a) comprises obtaining an image of an occluded portion of the vessel using magnetic resonance.

3. The method of claim 1 wherein the image includes an indication of a position of the recanalization device with respect to the occluded vessel.

4. The method of claim 3 wherein the image includes an indication of a spatial orientation of the recanalization device with respect to the occluded vessel.

5. The method of claim 1 wherein the image includes an image of the recanalization device.

6. The method of claim 1 wherein recanalizing step (c) comprises:
(c)(i) providing an electrical conductor having a substantially uninsulated distal tip;
(c)(ii) disposing the conductor in the occluded vessel with the distal tip proximate the occlusion; and
(c)(iii) applying an electrical current to the conductor such that the distal tip of the conductor creates heat.

7. The method of claim 6 wherein the electrical current applied to the conductor is a radio frequency current.

8. The method of claim 1 wherein obtaining step (a) comprises:
(a)(i) receiving a magnetic resonance signal with an external receiver located external to the body of the subject;
(a)(ii) generating a map image of the occluded vessel using the signal received by the external receiver;
(a)(iii) receiving a magnetic resonance signal with a first internal antenna positioned within the body of the subject, proximate to the occluded vessel; and
(a)(iv) locally enhancing the map image of the occluded vessel using the signal received by the first internal antenna.

9. The method of claim 8 wherein receiving step (a)(iii) comprises receiving a magnetic resonance signal with a first internal antenna that is integral with the recanalization device.

10. The method of claim 8 wherein receiving step (a)(iii) comprises receiving a magnetic resonance signal with a first internal antenna that is integral with equipment deployed in the vessel to assist in the delivery of the recanalization device to the occlusion.

11. The method of claim 10 wherein the antenna is integral with a guidewire deployed in the vessel to assist in the delivery of the recanalization device to the occlusion.

12. The method of claim 10 wherein the antenna is integral with a catheter deployed in the vessel to assist in the delivery of the recanalization device to the occlusion.

13. The method of claim 8 further comprising a step (a)(v) of:
(a)(v) calculating a position of the recanalization device based upon the magnetic resonance signal received by the first internal antenna.

14. The method of claim 13 further comprising a step (a)(vi) of:
(a) (vi) generating an integrated image of the occluded vessel based upon the map image, the locally enhanced image, and the calculated position of the recanalization device.

15. The method of claim 14 wherein the integrated image comprises a three-dimensional rendering showing the recanalization device and the occluded vessel.

16. The method of claim 14 wherein generating step (a)(iv) comprises:
(a)(vi)(A) generating an integrated image of the occluded vessel based upon the map image and the locally enhanced image; and
(a)(vi)(B) superimposing a symbol on the integrated image at a position representing the calculated position of the recanalization device.

17. The method of claim 8 wherein the magnetic resonance signals comprise radio frequency signals that are representative of the magnetic resonance of atomic particles in a vicinity proximate to the corresponding antenna.

18. The method of claim 8 wherein the first internal antenna comprises an elongated receiver coil having a pair of elongated electrical conductors that are electrically insulated from each other, each conductor having a distal end, the distal ends of the conductors being electrically coupled to each other, and wherein receiving step (a)(iii) comprises positioning the distal ends of the conductors proximate the occlusion.

19. The method of claim 18 further comprising steps of:
(a)(v) receiving a magnetic resonance signal with a second internal antenna comprising first and second elongated electrical conductors, the conductors being electrically insulated from each other and having spaced-apart distal ends, wherein the spaced-apart distal ends are positioned proximate the occlusion to receive the magnetic resonance signal; and
(a) (vi) locally enhancing the map image of the occluded vessel using the magnetic resonance signal received by the second internal antenna.

20. The method of claim 19 wherein the second internal antenna comprises a coaxial cable including the first and second conductors in a coaxial arrangement.

21. The method of claim 19 wherein the second internal antenna comprises a guidewire deployed in the vessel to assist in the delivery of the recanalization device to the occlusion.

22. The method of claim 19 wherein the first internal antenna comprises a catheter deployed in the vessel to assist in the delivery of the recanalization device to the occlusion.

23. The method of claim 8 wherein the first internal antenna comprises first and second elongated electrical conductors that are electrically insulated from each other, each conductor having a distal end, the distal ends of the conductors being electrically coupled to each other via a coil comprised of a helically wound electrical conductor, and wherein receiving step (a) (iii) comprises positioning the coil proximate the occlusion.

24. The method of claim 8 wherein the first internal antenna comprises first and second elongated electrical conductors, the conductors being electrically insulated from each other and having spaced-apart distal ends, and wherein receiving step (a)(iii) comprises positioning the distal ends of the conductors proximate the occlusion.

25. The method of claim 24 wherein the first internal antenna comprises a coaxial cable including the first and second conductors in a coaxial arrangement.

26. The method of claim 24 wherein the first conductor of the first internal antenna is adapted to function as an ablation wire in addition to its role in receiving the magnetic resonance signal, wherein the first conductor has a substantially uninsulated distal tip and wherein recanalizing step (c) comprises:
(c)(i) disposing the first conductor of the first internal antenna in the occluded vessel with the distal tip proximate the occlusion; and
(c)(ii) applying an electrical ablation current to the first conductor such that the distal tip of the first conductor vaporizes the substance forming the occlusion.

27. The method of claim 26 wherein the first conductor of the first internal antenna is couplable to a magnetic resonance imaging system adapted to produce the image of the occluded vessel and wherein the first conductor of the first internal antenna is further couplable to an ablation power supply adapted to apply the electrical ablation current to the first conductor.

28. The method of claim 27 further comprising a step (d) of:
(d) selectably switching the first conductor of the first internal antenna between the magnetic resonance imaging system and the ablation power supply.

29. The method of claim 26 wherein the first internal antenna comprises a coaxial cable including the first and second conductors in a coaxial arrangement, the first conductor being the center conductor of the coaxial cable.

30. The method of claim 8 wherein receiving step (a)(i) and generating step (a)(ii) are performed prior to guiding step (b) and recanalizing step (c).

31. The method of claim 30 wherein receiving step (a)(iii) and locally enhancing step (a)(iv) are performed real-time during the performance of guiding step (b) and recanalizing step (c).

32. The method of claim 8 wherein locally enhancing step (a)(iv) comprises:
(a)(iv)(A) generating a local image of the occluded vessel using the signal received by the first internal antenna; and
(a)(iv)(B) superimposing the local image on the map image of the occluded vessel generated using the signal received by the external receiver.

33. The method of claim 1 wherein obtaining step (a) comprises:
(a)(i) receiving a magnetic resonance signal with an external receiver located external to the body of the subject;
(a)(ii) generating a map image of the occluded vessel using the signal received by the external receiver;
(a)(iii) receiving a magnetic resonance signal with a first internal antenna positioned within the body of the subject, proximate to the occluded vessel; and
(a)(iv) generating a local image of the occluded vessel using the signal received by the first internal antenna.

34. The method of claim 33 wherein receiving step (a)(iii) comprises receiving a magnetic resonance signal with a first internal antenna that is integral with the recanalization device.

35. The method of claim 33 wherein receiving step (a)(iii) comprises receiving a magnetic resonance signal with a first internal antenna that is integral with equipment deployed in the vessel to assist in the delivery of the recanalization device to the occlusion.

36. The method of claim 35 wherein the antenna is integral with a guidewire deployed in the vessel to assist in the delivery of the recanalization device to the occlusion.

37. The method of claim 35 wherein the antenna is integral with a catheter deployed in the vessel to assist in the delivery of the recanalization device to the occlusion.

38. The method of claim 33 further comprising a step (a)(v) of:
(a)(v) generating an integrated image of the occluded vessel by combining the map image generated in generating step (a)(ii) and the local image generated in generating step (a)(iv).

39. The method of claim 38 wherein generating step (a)(v) comprises superimposing the local image on the map image.

40. The method of claim 33 further comprising a step (a) (v) of:
(a)(v) calculating a position of the recanalization device based upon the magnetic resonance signal received by the first internal antenna.

41. The method of claim 40 further comprising a step (a) (vi) of:
(a)(vi) generating an integrated image of the occluded vessel based upon the map image generated in generating step (a)(ii), the local image generated in generating step (a)(iv) and the calculated position of the recanalization device.

42. The method of claim 41 wherein the integrated image comprises a three-dimensional rendering showing the recanalization device and the occluded vessel.

43. The method of claim 41 wherein generating step (a)(vi) comprises:
(a)(vi)(A) generating an integrated image of the occluded vessel based upon the map image and the local image; and
(a)(vi)(B) superimposing a symbol on the integrated image at a position representing the calculated position of the recanalization device.

44. The method of claim 33 wherein the magnetic resonance signals comprise radio frequency signals that are representative of the magnetic resonance of atomic particles in a vicinity proximate to the corresponding antenna.

45. The method of claim 33 wherein the first internal antenna comprises an elongated receiver coil having a pair of elongated electrical conductors that are electrically insulated from each other, each conductor having a distal end, the distal ends of the conductors being electrically coupled to each other, and wherein receiving step (a)(iii) comprises positioning the distal ends of the conductors proximate the occlusion.

46. The method of claim 45 further comprising steps of:
(a)(v) receiving a magnetic resonance signal with a second internal antenna comprising first and second elongated electrical conductors, the conductors being electrically insulated from each other and having spaced-apart distal ends, wherein the spaced-apart distal ends are positioned proximate the occlusion to receive the magnetic resonance signal; and
(a) (vi) generating a local image of the occluded vessel using the magnetic resonance signal received by the second internal antenna.

47. The method of claim 46 wherein the second internal antenna comprises a coaxial cable including the first and second conductors in a coaxial arrangement.

48. The method of claim 46 wherein the second internal antenna comprises a guidewire deployed in the vessel to assist in the delivery of the recanalization device to the occlusion.

49. The method of claim 48 wherein the first internal antenna comprises a catheter deployed in the vessel to assist in the delivery of the recanalization device to the occlusion.

50. The method of claim 43 further comprising a step (a)(vii) of:
(a)(vii) generating an integrated image of the occluded vessel by combining the map image generated in generating step (a)(ii), the local image generated in generating step (a)(iv) and the image generated in imaging step (a)(vi).

51. The method of claim 33 wherein the first internal antenna comprises first and second elongated electrical conductors that are electrically insulated from each other, each conductor having a distal end, the distal ends of the conductors being electrically coupled to each other via a coil comprised of a helically wound electrical conductor, and wherein receiving step (a)(iii) comprises positioning the coil proximate the occlusion.

52. The method of claim 33 wherein the first internal antenna comprises first and second elongated electrical conductors, the conductors being electrically insulated from each other and having spaced-apart distal ends, and wherein receiving step (a)(iii) comprises positioning the distal ends of the conductors proximate the occlusion.

53. The method of claim 52 wherein the first conductor of the first internal antenna is adapted to function as an ablation wire in addition to its role in receiving the magnetic resonance signal, wherein the first conductor has a substantially uninsulated distal tip and wherein recanalizing step (c) comprises:
(c)(i) disposing the first conductor of the first internal antenna in the occluded vessel with the distal tip proximate the occlusion; and
(c)(ii) applying an electrical ablation current to the first conductor such that the distal tip of the first conductor vaporizes the substance forming the occlusion.

54. The method of claim 53 wherein the first conductor of the first internal antenna is couplable to a magnetic resonance imaging system adapted to produce the image of the occluded vessel and wherein the first conductor of the first internal antenna is further couplable to an ablation power supply adapted to apply the electrical ablation current to the first conductor.

55. The method of claim 54 further comprising a step (d) of:
(d) selectably switching the first conductor of the first internal antenna between the magnetic resonance imaging system and the ablation power supply.

56. The method of claim 55 wherein the first internal antenna comprises a coaxial cable including the first and second conductors in a coaxial arrangement, the first conductor being the center conductor of the coaxial cable.

57. The method of claim 52 wherein the first internal antenna comprises a coaxial cable including the first and second conductors in a coaxial arrangement.

58. The method of claim 33 wherein receiving step (a)(i) and generating step (a)(ii) are performed prior to guiding step (b) and recanalizing step (c).

59. The method of claim 58 wherein receiving step (a)(iii) and generating step (a)(iv) are performed real-time during the performance of guiding step (b) and recanalizing step (c).

60. An apparatus for imaging an occluded vessel in a subject, comprising:
a magnetic field generator adapted to establish a magnetic field on the subject;
a magnetic field gradient generator adapted to establish gradients in the magnetic field;
a radio frequency (RF) signal generator adapted to emit pulsed RF signals to at least the occluded vessel of the subject;
an external RF receiver adapted to be positioned external to the body of the subject, to receive RF signals emitted from the subject in response to the RF pulses and to provide an output signal in response to the received signals;
a first internal RF antenna adapted to be positioned in the occluded vessel proximate the occlusion, to receive RF signals emitted from the subject in response to the RF pulses and to provide an output signal in response to the received signals;
a controller adapted to receive and process the output signals from the external and internal RF antennas and to produce magnetic resonance (MR) information related thereto; and
a visual display adapted to receive the MR information produced by the processor and to display the MR information as an image of the occluded vessel.

61. The apparatus of claim 60 wherein the first internal RF antenna is associated with a recanalization device adapted to be positioned in the vessel proximate the occlusion and to recanalize the occluded vessel.

62. The apparatus of claim 61 wherein the first internal RF antenna is integral with the recanalization device.

63. The apparatus of claim 61 wherein the internal RF antenna is integral with equipment adapted to be deployed in the vessel to assist in the delivery of the recanalization device to the occlusion.

64. The apparatus of claim 63 wherein the first internal RF antenna is integral with a guidewire adapted to be deployed in the vessel to assist in the delivery of the recanalization device to the occlusion.

65. The apparatus of claim 63 wherein the first internal RF antenna is integral with a catheter adapted to be deployed in the vessel to assist in the delivery of the recanalization device to the occlusion.

66. The apparatus of claim 61 wherein the controller is adapted to calculate the position of the recanalization device based upon the output signals from the first internal RF antenna.

67. The apparatus of claim 66 wherein the visual display is adapted to receive the position information calculated by the controller and to display the position of the recanalization device with respect to the occluded vessel.

68. The apparatus of claim 67 wherein the visual display is adapted to superimpose a symbol on the image of the occluded vessel at a position representing the calculated position of the recanalization device.

69. The apparatus of claim 60 wherein the first internal RF antenna comprises an elongated receiver coil having a pair of elongated electrical conductors that are electrically insulated from each other, each conductor having a distal end, the distal ends of the conductors being electrically coupled to each other and adapted to be positioned proximate the occlusion.

70. The apparatus of claim 69 further comprising a second internal RF antenna comprising first and second elongated electrical conductors, the conductors being electrically insulated from each other and having spaced-apart distal ends, wherein the spaced-apart distal ends are adapted to be positioned proximate the occlusion to receive RF signals emitted from the subject, the second internal RF antenna adapted to provide an output signal in response to the received signals.

71. The apparatus of claim 70 wherein the second internal RF antenna comprises a coaxial cable including the first and second conductors in a coaxial arrangement.

72. The apparatus of claim 70 wherein the second internal RF antenna comprises a guidewire deployed in the vessel to assist in the delivery of the recanalization device to the occlusion.

73. The apparatus of claim 72 wherein the first internal RF antenna comprises a catheter deployed in the vessel to assist in the delivery of the recanalization device to the occlusion.

74. The apparatus of claim 60 wherein the first internal RF antenna comprises first and second elongated electrical conductors that are electrically insulated from each other, each conductor having a distal end, the distal ends of the conductors being electrically coupled to each other via a coil comprised of a helically wound electrical conductor, the coil adapted to be positioned proximate the occlusion.

75. The apparatus of claim 60 wherein the first internal RF antenna comprises first and second elongated electrical conductors, the conductors being electrically insulated from each other and having spaced-apart distal ends adapted to be positioned proximate the occlusion.

76. The apparatus of claim 75 wherein the first internal RF antenna comprises a coaxial cable including the first and second conductors in a coaxial arrangement.

77. The apparatus of claim 75 wherein the first conductor of the first internal RF antenna is adapted to function as an ablation wire in addition to its role in receiving the magnetic resonance signal, wherein the first conductor has a substantially uninsulated distal tip adapted to be positioned proximate the occlusion and wherein the first conductor is adapted to receive and conduct an electrical ablation current such that the distal tip of the conductor vaporizes the substance forming the occlusion.

78. The apparatus of claim 77 wherein the first conductor of the first internal RF antenna is couplable to an ablation power supply adapted to apply the electrical ablation current to the first conductor.

79. The apparatus of claim 78 further comprising a switch adapted to selectably switch the first conductor of the first internal RF antenna between the controller and the ablation power supply.

80. The apparatus of claim 77 wherein the first internal RF antenna comprises a coaxial cable including the first and second conductors in a coaxial arrangement, the first conductor being the center conductor of the coaxial cable.

81. The apparatus of claim 60 wherein the controller is adapted to receive the external RF receiver output signal and to produce a first set of MR information related thereto, and to receive the internal RF antenna output signal and to produce a second set of MR information related thereto, and wherein the visual display is adapted to provide a first view of the occluded vessel based on the first set of MR information and to provide a second view of the occluded vessel based on the second set of MR information.

82. The apparatus of claim 81 wherein the visual display is adapted to integrate the first and second views of the occluded vessel to produce an integrated image of the occluded vessel.

83. The apparatus of claim 82 wherein the controller is adapted to calculate the position of the recanalization device based upon the output signals from the first internal RF antenna and wherein the visual display is adapted to receive the position information calculated by the controller and to display the position of the recanalization device with respect to the occluded vessel in the integrated image.

84. The apparatus of claim 83 wherein the visual display is adapted to superimpose a symbol on the integrated image of the occluded vessel at a position representing the calculated position of the recanalization device.

85. The apparatus of claim 83 wherein the integrated image comprises a three-dimensional rendering showing the recanalization device and the occluded vessel.
